(19) United States
(12) Patent Application Publication
(10) Pub. No.: US 2018/0243025 A1
(43) Pub. Date: Aug. 30, 2018

(54) Operating Room Fire Prevention and Electrocautery Safety Device

(71) Applicant: THE REGENTS OF THE UNIVERSITY OF COLORADO, A BODY CORPORATE, Denver, CO (US)

(72) Inventors: David Smits, Denver, CO (US); Eric Hink, Louisville, CO (US)

(21) Appl. No.: 15/753,931

(22) PCT Filed: Aug. 19, 2016
(86) PCT No.: PCT/US16/47821

§ 371 Date: Feb. 20, 2018

Related U.S. Application Data
(60) Provisional application No. 62/207,616, filed on Aug. 20, 2015.

(51) Int. Cl. A61B 18/12; A61B 18/14; A62C 3/00; G01N 33/00

(52) U.S. Cl. CPC A61B 18/1233; A61B 18/1442; A61B 2018/00595; G01N 33/0031; A62C 3/00

(57) ABSTRACT

This invention is in the field of surgical tools for the prevention of operating room fires. The invention relates to a system and methods which minimizes the threat to the health and safety of surgical patients and medical staff resulting from lapses in human judgment that result in inadvertent violations of conventional fire prevention protocols.

### CROSS-REFERENCE TO RELATED APPLICATIONS

The present application claims the benefit of U.S. Provisional Patent Application No. 62/207,616, filed on Aug. 20, 2015, which is incorporated herein by reference.

### FIELD OF THE INVENTION

This invention is in the field of surgical tools for the prevention of operating room fires. The invention relates to a system and methods which minimizes the threat to the health and safety of surgical patients and medical staff resulting from lapses in human judgment that result in inadvertent violations of conventional fire prevention protocols.

### BACKGROUND OF THE INVENTION

Operating room fires and the hazards associated therewith are well known in the art. In this regard, hundreds of operating room fires arise annually during the performance of a variety of surgical procedures, resulting in dramatic tragedy to the affected patients and/or health care workers. Although not all such cases are reported due to liability issues, occasionally high profile cases have been publicized to this effect. In addition to the high fatality, several burn complications that result in lifelong suffering of the patients necessitates a solution for this problem.

Notwithstanding the implementation of a variety of safeguards to prevent operating room fires, even the best practices are not effective to substantially reduce the risk of operating room fires. What is needed is a system or method currently available that enables high-risk surgical equipment, and in particular electrosurgical instruments such as electrocautery pin knives, lasers and the like, to be effectively utilized only in the absence of oxygen-enriched environments therefore effectively eliminating the potential for such elements to create a fire hazard. There is likewise substantially lacking in the art any type of system and method for reducing the risk of operating room fires that can be readily integrated as part of an existing electrosurgical device and oxygen source, and in particular an electrocautery cutting apparatus that can be utilized per conventional electrocautery instruments and be utilized per conventional electrosurgical instruments for use in performing a wide variety of surgical procedures in conjunction with conventional oxygen sources, such as oxygen tanks or other enriched oxygen sources. There is likewise no system of preventing unintentional combustion with electrosurgical instruments in the presence of unsafe levels of oxygen in the operative field, by prevention of operation of said electrosurgical instruments. There is a need for a system and method that is of simple construction, low cost, very safe to utilize and can be constructed utilizing well-known, commercially available materials to prevent inadvertent fires through lapses in conventional fire prevention protocols.

### SUMMARY OF THE INVENTION

This invention is in the field of surgical tools for the prevention of operating room fires. The invention relates to a system and methods which minimizes the threat to the health and safety of surgical patients and medical staff resulting from lapses in human judgment that result in inadvertent violations of conventional fire prevention protocols. The present invention specifically addresses and alleviates the above-identified deficiencies in the art.

In one embodiment, the invention relates to an oxygen sensor system for minimizing the outbreak of an operating room fire comprising: a) an electrocautery device having a proximal end and a distal end for electrically cauterizing tissue; b) an oxygen source comprising an in-line oxygen flow sensor; and c) a relay switch in electrical communication with said oxygen flow sensor and said electrocautery device. In one embodiment, said relay switch is in electrical communication with a control unit. In one embodiment, said electrocautery device comprises monopolar cautery. In one embodiment, said electrocautery device comprises bipolar cautery. In one embodiment, said sensor system with said relay switch comprises a single device with circuitry for both bipolar and monopolar cautery. In one embodiment, the system further comprises a switch to toggle between both bipolar and monopolar cautery circuitry. In one embodiment, said system further comprises an indicator on the device signaling a closed cautery circuit. In one embodiment, said indicator is a visual indicator. In one embodiment, said visual indicator is an LED. In one embodiment, said indicator is a sonic indicator. In one embodiment, said indicator is a vibration indicator. In one embodiment, said system further comprises an integrated activation time delay in the relay switch after sensing a minimum threshold of oxygen flow to activate the relay. In one embodiment, said system further comprises additional oxygen concentration sensors integrated into the relay circuitry. In one embodiment, said sensors are clipped under or over surgical drapes monitoring buildup of oxygen concentration. In one embodiment, said at least one sensor detects concentration above a minimum threshold of oxygen, the sensor deactivates the relay, thus opening the cautery circuit not allowing use of the cautery devices. In one embodiment, said minimum threshold of oxygen comprises 25%. In one embodiment, said system is configured to interface with the internal components of a commercially available device. In one embodiment, said commercially available device comprises a electrocautery generator or electrosurgical generator. In one embodiment, said electrocautery device interfaces with an electrocautery generator and is separately powered through an outlet. In one embodiment, said electrocautery device may interface with an electrocautery generator through an auxiliary port. In one embodiment, said electrocautery device may interface with an electrocautery generator through an expansion port. In one embodiment, said electrocautery device may interface with an electrocautery generator through an existing port. In one embodiment, said electrocautery device utilizes an auxiliary port on an existing electrocautery generator that both powers the device, and receives a signal from said flow sensor or control unit to switch an internal relay or control unit and shut off the ability to cauterize circuits, including both monoploar and bipolar circuits. In one embodiment, said electrocautery device is integrated completely into an electrocautery generator. In one embodiment, the invention may further include an indicator on the device signaling a closed cautery circuit, indicating to the surgeon it is safe to use. In one embodiment, the invention further comprises an indicator that cautery is disabled and one cannot use cautery. In one embodiment, the invention further comprises an indicator that shows the delay time. In one embodiment, said delay time is a number indicator. In one embodiment, the invention relates method of using the system described above.

In one embodiment, the invention relates to an oxygen sensor system for minimizing the outbreak of an operating room fire comprising: a) an electrocautery device having a proximal end and a distal end for electrically cauterizing tissue; b) an oxygen source comprising an in-line oxygen flow sensor; and c) a relay switch in electrical communication with said oxygen flow sensor and said electrocautery device. In one embodiment, said relay switch is in electrical communication with a control unit. In one embodiment, the invention relates to a method of using the system of described above. In one embodiment, the invention may be used for monopolar cautery, including monopolar outlets/plugs and circuitry (See FIG. 3 & FIG. 4). The invention may encompass a separate device or a single device with circuitry for both bipolar and monopolar cautery. In one embodiment, said system is configured to interface with the internal components of a commercially available device. In one embodiment, said commercially available device comprises a electrocautery generator or electrosurgical generator. In one embodiment, said electrocautery device interfaces with an electrocautery generator and is separately powered through an outlet. In one embodiment, said electrocautery device may interface with an electrocautery generator through an auxiliary port. In one embodiment, said electrocautery device may interface with an electrocautery generator through an expansion port. In one embodiment, said electrocautery device may interface with an electrocautery generator through an existing port. In one embodiment, said electrocautery device utilizes an auxiliary port on an existing electrocautery generator that both powers the device, and receives a signal from said flow sensor or control unit to switch an internal relay or control unit and shut off the ability to cauterize circuits, including both monoploar and bipolar circuits. In one embodiment, said electrocautery device is integrated completely into an electrocautery generator. In one embodiment, the invention may further include an indicator on the device signaling a closed cautery circuit, indicating to the surgeon it is safe to use. In one embodiment, the invention further comprises an indicator that cautery is disabled and one cannot use cautery. In one embodiment, the invention further comprises an indicator that shows the delay time. In one embodiment, said delay time is a number indicator. In one embodiment, the invention relates method of using the system described above.

In one embodiment, said indicator is a visual indicator. In one embodiment, said visual indicator is an LED. In one embodiment, said indicator is a sonic indicator. In one embodiment, the indicator is a vibration indicator. In one embodiment, the invention may further include an integrated time delay in the circuit to respond once the oxygen flow reaches a minimum threshold to activate the relay. In one embodiment, the time delay may allow time for oxygen in the surgical field to dissipate. In one embodiment, the invention may further include additional oxygen concentration sensors integrated into the relay circuitry. In one embodiment, the sensors are clipped under or over surgical drapes monitoring buildup of oxygen concentration. In one embodiment, when the sensor detects concentration above a minimum threshold of oxygen, the sensor deactivates the relay, thus opening the cautery circuit not allowing use of the cautery devices.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “patient” or “subject” refers to any living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses. Although described in particular as applicable to hospitals and medical practices treating human patients, the benefits of the invention apply to veterinarian practices as well; “patient” then may be a pet or other animal, as well as a human patient.

As used herein “prevention” or “preventing” as used herein, includes, but is not limited to: to substantially reduce the occurrence of operating room fires.

As used herein “operating room” is meant any hospital operating room or a doctor or other medical practitioner's office outfitted for surgery.

As used herein “oxygen-rich” or “oxygen source” or “O₂ source” means pure oxygen, oxygen enriched air, nitrous oxide or another gas delivered to a patient and capable of creating a region of heightened oxygen content on or around the patient. In some embodiments, an oxygen source may comprise an anesthesia machine.

As used herein, the term “proximal” refers to a location situated toward a point of origin (e.g., between a physician and the device).

As used herein, the term “distal” refers to a location situated away from a point of origin (e.g., the end of the device).

As used herein, the term “electrosurgical generator” or “electrocautery generator” are used interchangeably to refer to a device that may create heat in a surgical setting that may result in a potential fire, whether said device creates a heated probe be used for the cauterization of tissue in some applications or a device that uses radio frequency (RF) alternating current to heat the tissue by RF induced intracellular oscillation of ionized molecules that result in an elevation of intracellular temperature. In some instances, “electrosurgical generator” or “electrocautery generator” also refer to RF generators, ultrasound generators, or argon plasma generators. In some instances, electrocautery refers to the use of heat conduction from a probe heated to a glowing temperature by a direct current (much in the manner of a soldering iron). This may be accomplished by direct current from dry-cells in a penlight-type device. In some instances, electrosurgery, by contrast, uses radio frequency (RF) alternating current to heat the tissue by RF induced intracellular oscillation of ionized molecules that result in an elevation of intracellular temperature.

### DESCRIPTION OF THE FIGURES

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention.

FIG. 1 shows one embodiment of the device 54 wherein an O₂ flow sensor 58 is coupled to a relay switch 62. The O₂ flow sensor 58 is between the O₂ source 55 (tank) and patient's nasal cannula, mask, endotracheal tube, laryngeal mask airway, or other O₂ delivery device 59 and controls the relay switch 62. The relay switch 62 controls the on/off function of the cautery circuit 65. When O₂ is flowing, the cautery forceps or tip 63 will not turn on. When O₂ flow stops, the relay switch 62 closes allowing use of cautery 63, in one embodiment after a brief time delay built into the cautery circuitry.

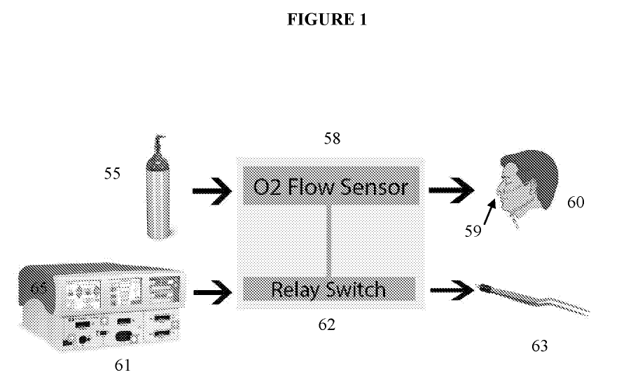

FIG. 2 shows one embodiment of the device 54 wherein the electrocautery device, forceps, or tip, 63 plugs into the relay switch device monopolar or bipolar female outlet 67 and the relay switch device 54 further connects through a relay switch device monopolar/bipolar male outlet 66 into electrocautery generator 61 and receives O₂ from the O₂ source (anesthesia machine or patient O₂ tank). The tubing from the O₂ source plugs into the device O₂ input 56, running through the O₂ flow sensor 58, and out the O₂ output 57, leading to the patient's nasal cannula, mask, endotracheal tube, laryngeal mask airway, or other O₂ delivery device 59.

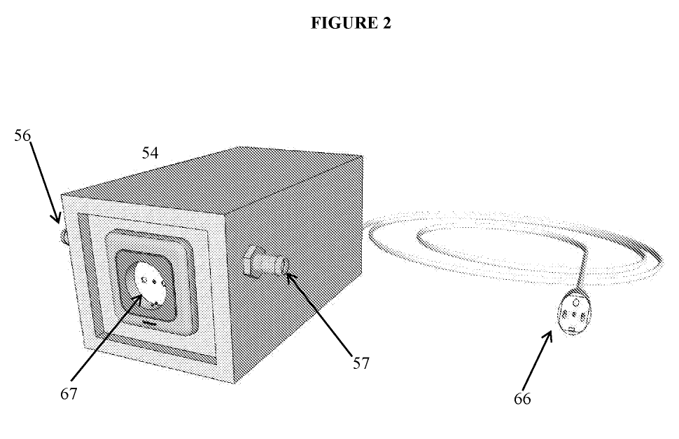

FIG. 3 shows a diagram of the operation of the device when the O₂ source is not flowing. When the O₂ source is not flowing, the relay is energized and the normally open (NO) switch is closed. When the relay is energized, the electrocautery circuit is closed and complete, but there is no O₂ source flowing. This prevents inadvertent oxygen ignition as the electrocautery circuit is closed and complete, but there is no O₂ source flowing.

FIG. 4 shows a diagram of the operation of the device when the O₂ source is flowing. When the O₂ source is flowing, the relay is not energized and the normally open (NO) switch is open. When the relay is not energized, the electrocautery circuit is open, and not complete. This prevents inadvertent oxygen ignition as the electrocautery circuit is closed and not complete.

FIG. 5 demonstrates how O₂ concentration at the surgery site will increase at various flow rates based on the mathematical model described in herein.

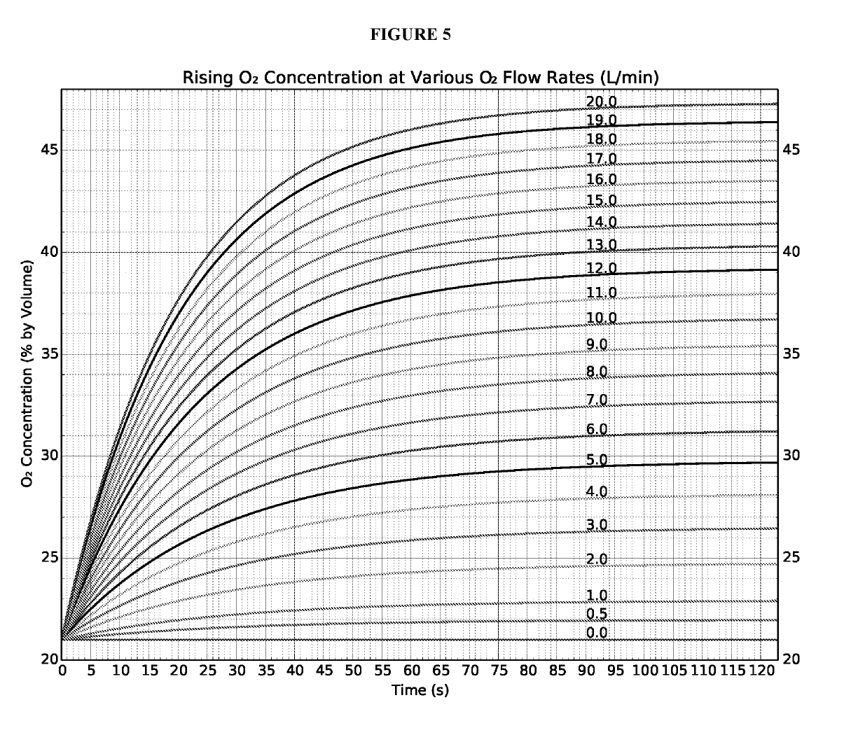

FIG. 6 demonstrates how O₂ concentration at the surgery site will decrease based on the mathematical model described in herein.

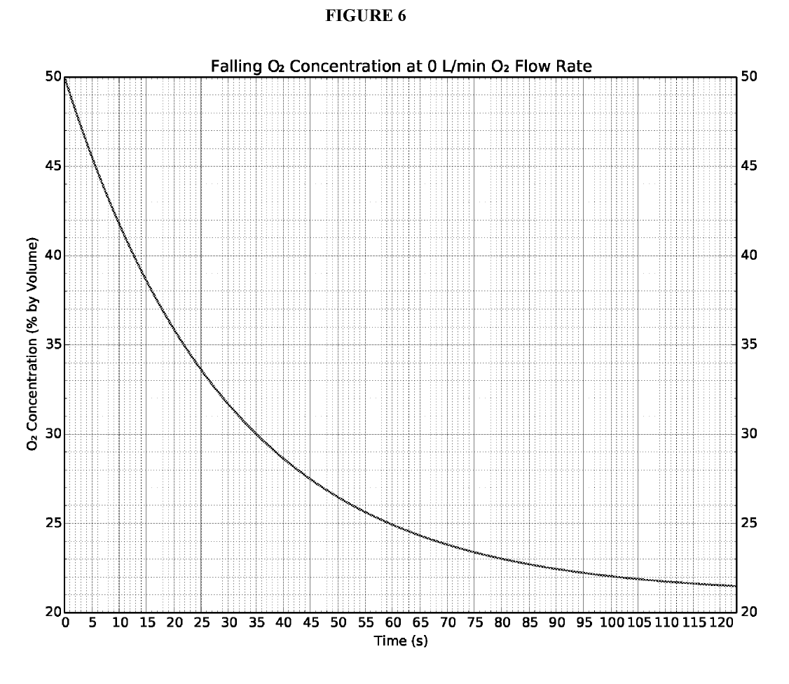

FIG. 7 shows the variables considered in the system.

FIG. 8 shows the complete assembly front view of one embodiment of the current invention.

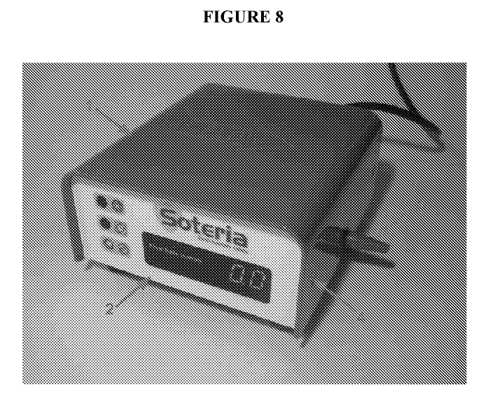

FIG. 9 shows the complete assembly back view of one embodiment of the current invention.

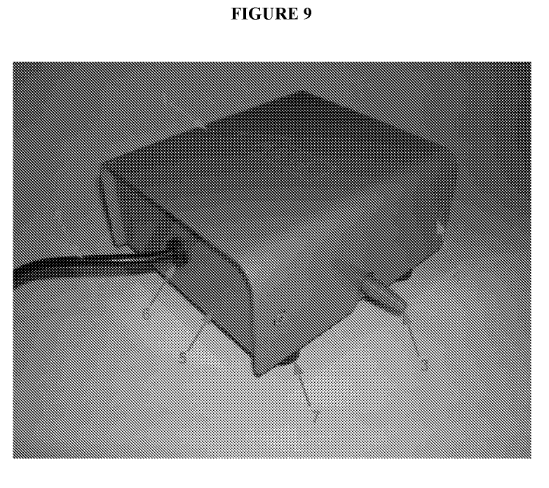

FIG. 10 shows the complete assembly bottom view of one embodiment of the current invention.

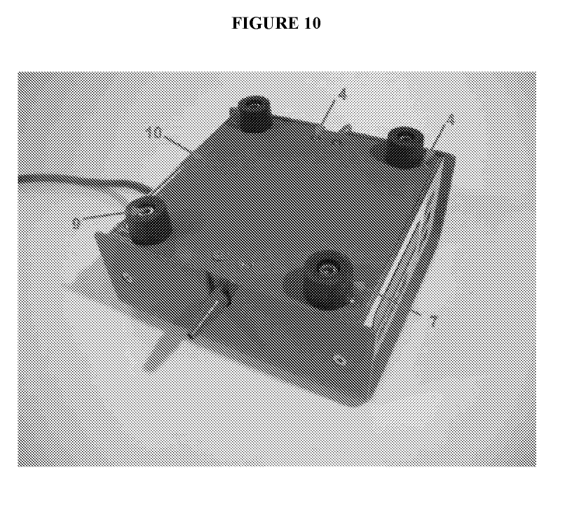

FIG. 11 shows an internal view of one embodiment of the current invention.

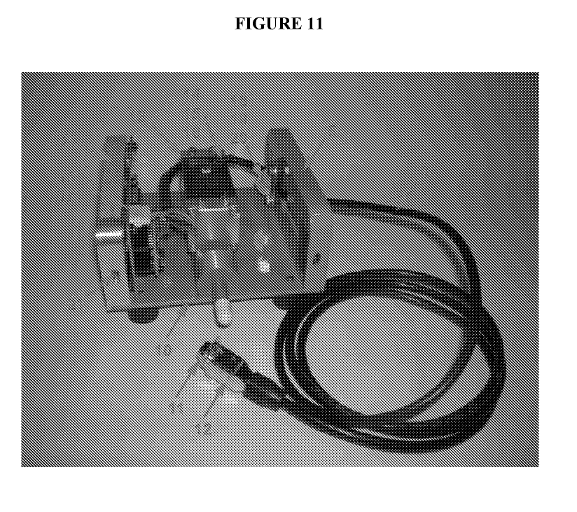

FIG. 12 shows a flow sensor assembly of one embodiment of the current invention.

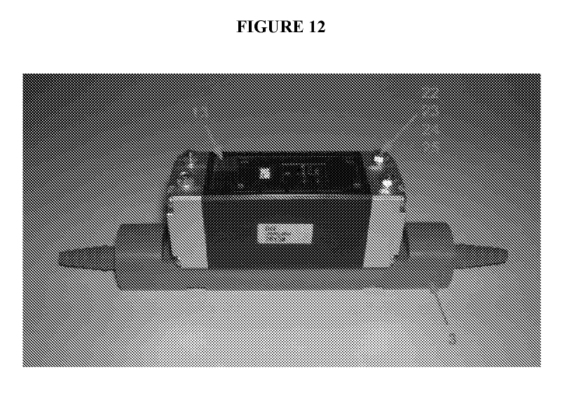

FIG. 13 shows a flow fitting.

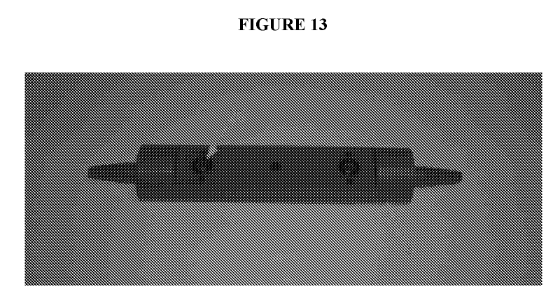

FIG. 14 shows a front panel front view of one embodiment of the current invention.

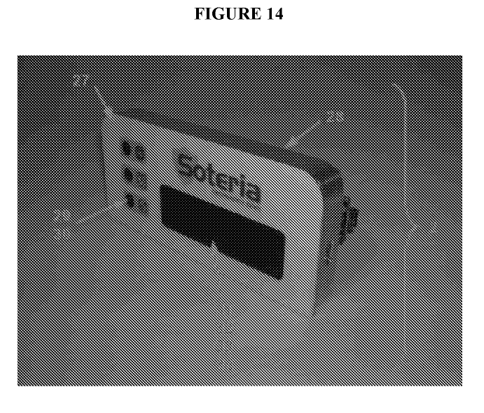

FIG. 15 shows a front panel back view of one embodiment of the current invention.

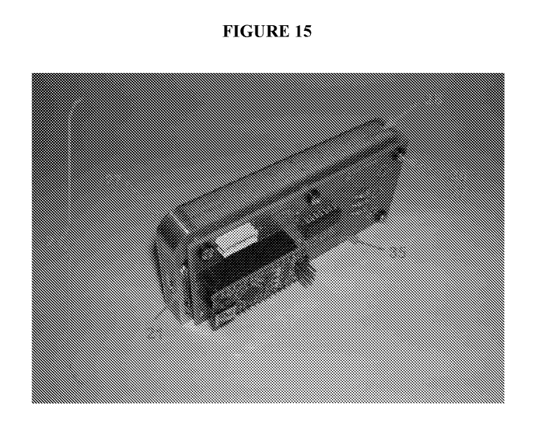

FIG. 16 shows a circuit board front view of one embodiment of the current invention.

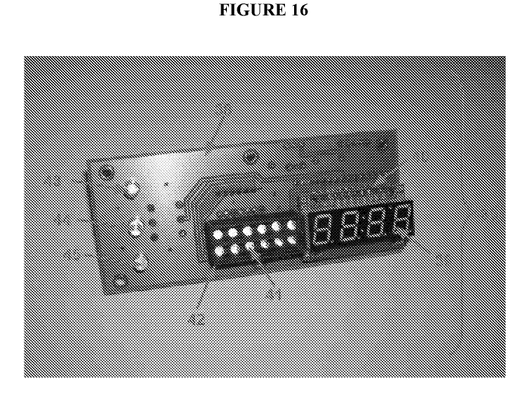

FIG. 17 shows a circuit board back view of one embodiment of the current invention.

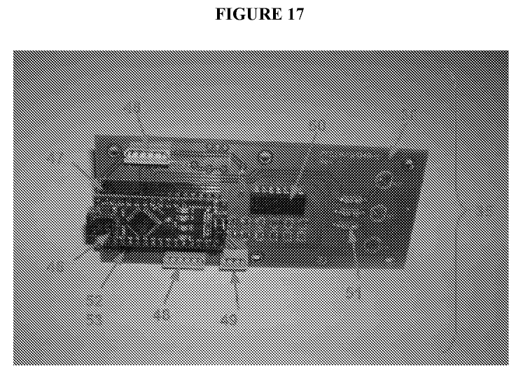

FIG. 18 shows an electrical schematic of one embodiment of the current invention. It should be noted that P4 connects to expansion port on a Covidien Force FX electrosurgical generator and J2 is a spare I/O connector for diagnostic use or future expansion. In one embodiment, P4 may connect to an existing, expansion, or auxiliary port of an electrocautery generator.

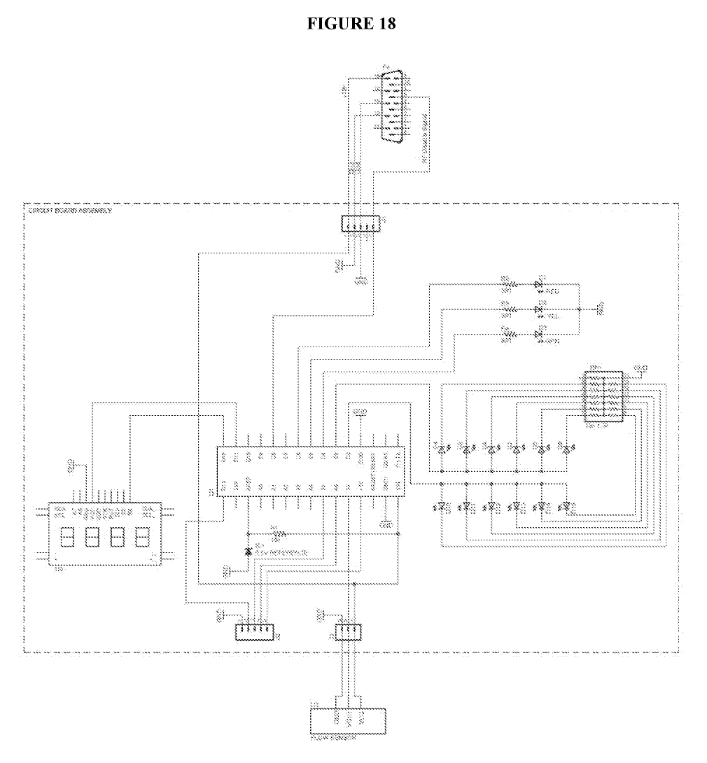

FIG. 19 shows a software description flow chart for one embodiment of the current invention.

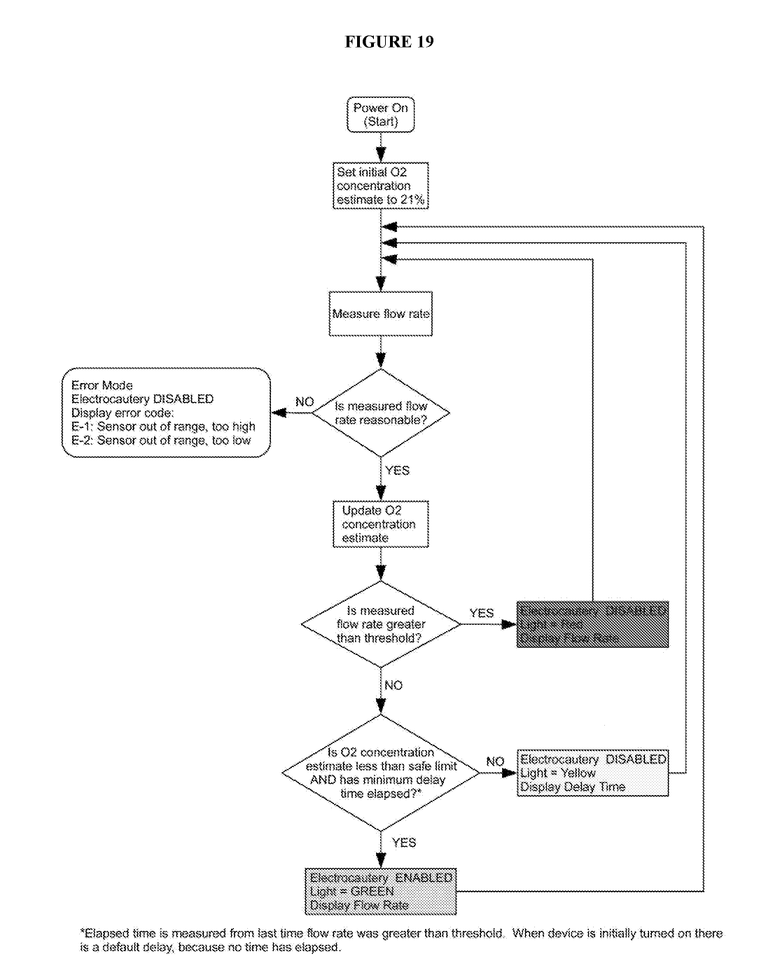

### DETAILED DESCRIPTION OF THE INVENTION

This invention is in the field of surgical tools for the prevention of operating room fires. The invention relates to a system and methods which minimizes the threat to the health and safety of surgical patients and medical staff resulting from lapses in human judgment that result in inadvertent violations of conventional fire prevention protocols.

### Operating Room Fires

There are 550-650 OR fires each year. The surgical equipment, including electrocautery devices, is believed to be an ignition source in 90% of these cases. Oxygen is oxidizer source in 95% of these cases. Majority (83%) occur during monitored anesthesia care (MAC) or regional anesthesia (RA) with 99% using supplemental O₂ by nasal cannula or mask. Of the incidents, 97% are during head, neck, upper chest surgery (64% plastics).

Although a number of factors are known to contribute to the cause of operating room fires, such as flammable materials including alcohol prepping agents and gowns, the most significant etiologic factor for operating room fires is the combination of heat generated from electrically powered surgical equipment in the presence of oxygen. With respect to the electrically powered surgical equipment, it is well known that a variety of surgical equipment and in particular electrocautery surgical instruments and lasers are known to emit substantial heat. Moreover, the tip of the electrocautery knife, due to the electrical current passing through, or the beam of the laser device is exceptionally prone to ignite a fire. A high concentration of oxygen and other flammable gases are also typically present, particularly during surgical procedures involving the head and neck insofar as oxygen tends to build beneath the surgical drapes, in or around the surgical field, or in the oropharyngeal cavity, which thus are operative to create an oxygen-enriched combustible atmosphere. Under such conditions, materials that are not considered flammable in normal circumstances can easily ignite with the resultant fire burning more violently and/or at higher temperatures. Although there are many methods of delivery of oxygen to the patient, there is currently no means of monitoring any oxygen leaking throughout the procedure.

Despite the well-known hazards associated with performing surgery under such conditions, however, there has not heretofore been any effective type of system or method that is operative to minimize the potential outbreak of operating room fires or even reduce the impact of the fire when it erupts. In this regard, the best safety practices currently in use merely involve taking precautionary measures and typically consist of nothing more than making efforts to minimize the buildup of oxygen and nitrous oxide, activating electrosurgical and electrocautery units at lower power settings, and/or avoiding surgical draping. Additional precautions include turning equipment off when not in use or otherwise placing electrosurgical instruments in a safe location, such as a safety holster, when not in active use. Likewise recommended is the practice of allowing a certain amount of time, such a minute or more, to discontinue oxygen administration to the patient prior to the use of the electrosurgical instruments, lasers and the like. As such, constant vigilance and communication between the anesthesiologist in charge of oxygen delivery and the surgeon in charge of the electrosurgical instruments is necessary.

### Electrosurgical and Electrocautery Tools of the Art

### RF Generators

RF generators are the most widely used type of installed electrosurgical equipment. Often called Electrosurgical Units (ESUs), RF generators may feature a variety of different modes and settings. Surgeons use the terms “RF surgery” and “electrosurgery” often interchangeably, highlighting the ubiquity of RF generators. Monopolar and bipolar devices are the two major categories of instruments through which the current is delivered to the patient to achieve the desired effect. The principles behind all RF surgeries are the same: vaporization, desiccation, coagulation, and fulguration of tissue by applying electrical currents of various characteristics. Surgeons control the desired effect on tissue by utilizing specific accessories, adjusting generator settings, and handling the instruments in a precise and careful manner.

Most RF generators have three main modes of operation, enabled by generating “cutting,” “coagulation,” and “blended” currents. The cutting mode delivers alternating current at a continuous high-duty cycle, causing rapid heat generation, vaporization of water and other particles within cells, and tissue rupture and cleavage. The cutting effect observed in tissue results from the rapid heat generation from the delivered current, rather than any physical cutting. The coagulation mode only has a 6% duty cycle, meaning that the current is actually delivered to the target tissue about 6% of the total time. The coagulation mode also features a higher output voltage to maintain a fixed amount of output power. The alternate characteristics of the coagulation current favor tissue coagulation over tissue vaporization. The blended output features a mixture of the characteristics of the cut and coagulation currents. The blended mode often has a higher duty cycle than the coagulation current, while outputting a lower voltage to maintain steady power generation [1]. Most RF generators are highly compatible with a variety of instruments; they can be used in conjunction with both monopolar and bipolar instruments.

### Ultrasonic Generators

In contrast to RF electrosurgery, ultrasonic surgery involves the conversion of electrical energy to mechanical vibrations via transducers in instruments in order to dissect and coagulate tissue [2]. Like RF electrosurgery however, ultrasonic surgery can be indispensable for both open and laparoscopic procedures. Tips, blades, and shears deliver the mechanical vibrations necessary for incision, dissection, and coagulation.

### Argon Plasma Coagulation Generators

Unlike RF electrosurgery, argon plasma coagulation (APC) is confined to a handful of specialties and procedure types, mostly within flexible endoscopy and bronchoscopy. Its application has been steadily increasing in otolaryngologic and dermatologic procedures as well. APC involves the delivery of inert argon gas, the flow rate of which is carefully controlled by the APC generator. An electrical spark, sometimes delivered by an RF generator adjacent to the APC equipment, converts the argon gas into plasma, which delivers thermal energy to target tissue at shallow depths [3].

### Electrocautery Generators

Unlike monopolar or bipolar modes of electrosurgery, electrocautery does not involve the passing of current through the patient. Electrocautery does feature direct contact with heated instruments. A tip or electrode is resistively heated and subsequently applied to tissue for selective destruction or hemostasis. Electrocautery is suitable for patients with implanted electrical devices, such as cardiac pacemakers, and is routinely utilized for minor procedures by dermatologists, ophthalmologists, plastic surgeons, and urologists.

### Monopolar Instruments

Strictly speaking, all RF electrosurgical procedures are bipolar: the current is generated through an active electrode and returns through a return electrode. The intended path of the current through the patient differentiates monopolar instruments from bipolar instruments. In monopolar electrosurgery, the current delivered through the active electrodes travels through the body of the patient and returns through the return electrode padding that is attached to the side of patient that is opposite to the surgical site. Depending on the technique, electrode shape, electrode location, and generator settings, a surgeon can choose to separate, fulgurate, or desiccate tissue.

Most companies marketing monopolar instruments and accessories sell both reusable and disposable pencils and electrodes. Bovie Medical, for example, features reusable and disposable handles, as well as tips, blades, and electrodes in a variety of shapes to meet the need of different types of procedures. Typically, a type of electrode is inserted into a monopolar pencil, which may be activated and deactivated by push buttons or foot pedals connected to the generator. Monopolar instruments are widely used in most types of electrosurgery that require at least an initial incision.

### Bipolar Instruments

Bipolar instruments pass the current through the patient for very short distances to achieve dissection or coagulation: the electrical current is delivered and returned through the same hand piece. Bipolar instruments, which are often compatible with the same RF generators that power monopolar instruments, are indispensable for both open and key-hole procedures. Bipolar instruments do not require patient return electrodes and are more effective at tissue and vessel sealing than monopolar instruments. Traditional bipolar instruments, such as simple bipolar forceps, are capable of sealing vessels of a large range of diameters. For more precise control of sealing, thermal, and mechanical effects, surgeons utilize advanced bipolar instruments.

### Advanced Bipolar Instruments

Advanced bipolar instruments allow for finer control of vessel sealing at precise diameter intervals. Some examples of advanced bipolar instruments include Medtronic's LigaSure line of devices and Ethicon's Enseal brand of vessel sealers. These instruments rely on both heat generation and mechanical pressure to achieve the desired results. Advanced bipolar instruments interface with surgical generators, which can be configured to deliver relatively low amounts of voltage during procedures. Additionally, the energy delivery cycles to the instruments can be coupled with tissue sensing features on an advanced bipolar device to minimize thermal damage and enhance seal strength.

### Ultrasonic Instruments

Unlike monopolar and bipolar instruments, ultrasonic instruments utilize high-frequency mechanical vibrations to cut and coagulate tissue. Typically, a hand held transducer converts the electrical energy from a generator to ultrasonic vibrations, which are then transferred to the tip. The instruments at the tip of ultrasonic assemblies may include blades, scissors, and shears. These ultrasonic tips replicate much of the same functions found in RF instruments, such as cutting and coagulating tissue. Additionally, some manufacturers in ultrasonic surgery also market advanced ultrasonic vessel and tissue sealing instruments that are comparable to advanced bipolar instruments. Examples of advanced ultrasonic sealers include Ethicon's Harmonic Shears and Lotus's Vessel Welder. Surgeons concerned with reducing thermal damage may elect to use these instruments instead of bipolar devices.

### Argon Plasma Coagulation Instruments

APC instruments, interfaced with the argon plasma generator, deliver a jet of argon plasma to target tissue. In most APC procedures, a monopolar electrode inside an APC instrument ionizes the flowing argon gas surrounding it. APC instruments can either be rigid or flexible, depending on the application. The Germany-based firm Erbe markets flexible APC probes for endoscopic procedures in multiple tip configurations to allow the surgeon to maneuver the plasma jet to the right targets. Rigid APC instruments, on the other hand, are more useful in bronchoscopic interventions. Erbe markets rigid applicators compatible with either intermediate handles or directly with APC generators.

### Prior Art Devices

There are other systems and devices described in prior art related to addressing the problem of operating room fires.

One reference, U.S. application Ser. No. 10/940,330 [4] describes oxygen sensor systems that are effective in substantially reducing the risk of causing an operating room fire instigated by electrocautery surgical instruments and laser systems. The reference also describes an oxygen sensor system that substantially reduces, if not eliminates, the potential for an outbreak of an operating room fire by warning of unsafe levels of oxygen and disabling either the ignition source or the oxygen delivery. The reference also describes a variation where the oxygen sensor could be coupled with an automatic shutoff mechanism to the release valve of the oxygen delivery system. The system typically is integrated into the electrocautery surgical instruments and shuts off the instrument power at predetermined oxygen level. The reference also describes the use of an oxygen sensor tip placed separately from the electrocautery instrument in the operative field, or even just outside the source of the oxygen to assure against excessive leaks. While not directed to detecting an oxygen flow, the reference does describe cutting power to the electrocautery surgical instruments at predetermined oxygen levels at the site of the electrocautery surgical instruments. The reference does not describe a device or method of using an oxygen source flow sensor to disable potential ignition devices, such as electrocautery as a default.

Another reference, U.S. Pat. No. 7,296,571 [5] describes a safety interlock that disables instrumentalities (such as an electrical cautery device) capable of igniting a fire in the presence of oxygen and a fuel source or combustible material such as skin, hair and/or prep agents containing alcohol. Removal of, e.g., the cautery device is sensed by a sensor to disable an oxygen source supplying oxygen to a patient (via an electrical oxygen source cut-off valve) and set a timer. Electrical activation of the cautery device only is permitted upon timing-out of the timer, which timing out is set for a time when oxygen will have cleared from the patient's immediate surroundings. A further safety feature is provided, by the same or another timer, which determines the length of time during which the patient has been deprived of oxygen. After a short duration, then, the cautery (or other) device is denied electrical power and oxygen is permitted to flow again to the patient. The reference does not describe a device or method of using an oxygen source flow sensor to disable potential ignition devices, such as electrocautery.

Another reference, U.S. patent application Ser. No. 10/949,856 [6], describes a system for prevention of fires in operating rooms from electrocautery and laser systems comprising an electrocautery instrument having a shielding gas that is expelled from the distal-most end to prevent the cautery tip spark from coming into contact with an oxygen-enriched environment that may spark a fire. The reference also describes an oxygen sensor placed at the distal tip of the electrocautery device, which may initiate inert gas flow depending on the detected oxygen concentration. The reference further describes a heat sensor strip, such as thermister or thermocouple device, operatively attached to the distal ends of a ventilator apparatus utilized in conjunction when the surgical procedure is performed, that is coupled with an oxygen release valve and/or the electrocautery device such that the apparatus automatically turns off oxygen delivery and/or electrosurgical device in the event of reaching predetermined temperature level. The reference also describes a temperature sensitive alarm to warn the anesthesiologist from continuing the flow of oxygen or nitrous oxide, as well as to warn the surgeon to refrain from using energy transmission from the electrosurgical instrument. Moreover, it is contemplated that the heat sensor device can be operatively coupled with the inert gas flushing mechanism and thus designed to be automatically turned on when preset temperature levels are recorded. The reference does not describe a device or method of using an oxygen source flow sensor to disable potential ignition devices, such as electrocautery.

Another reference, U.S. Pat. No. 8,789,524 [7], describes a medical gas delivery system that includes concentration of the medical gas dependent upon the precise control of the flow rate of each medical gas out of the pressurized medical gas sources, which is limited by minimum flow rates required by the flow sensors. The gases described include oxygen. The reference also describes a combination of at least two electronic medical gas-mixing systems. While the reference does describe an oxygen source flow sensor, reference does not describe a device or method of using an oxygen source flow sensor to disable potential ignition devices, such as electrocautery.

Another reference, U.S. patent application Ser. No. 12/535,103 [8], describes an electronic control system and perfusion fluid loop for an organ transporter which among other components includes an array of sensors. The sensors can include, for example, a pressure transducer, an oxygen sensor, a flow rate or pressure sensor, a delivery temperature sensor, an oxygen flow sensor, a reservoir pressure sensor, a reservoir temperature sensor, and organ fluid output sensors, (one or more metabolite sensors), (potassium), (sodium), and (oxygen concentration). While the reference does describe an oxygen source flow sensor, reference does not describe a device or method of using an oxygen source flow sensor to disable potential ignition devices, such as electrocautery.

Another reference, U.S. patent application Publication Ser. No. 10/295,141 [9], describes a medical ventilator. The ventilator includes a pneumatic system with a gas flow generator that draws air from air inlet for delivery to the patient. The system includes an oxygen flow sensor that measures the flow of oxygen out of oxygen flow valve. This measurement is used for closed loop control of oxygen flow valve and also to compute flow/volume delivered to the patient. The oxygen sensor provides a measurement of the oxygen concentration of gas being blended by the oxygen and blower valves. The inhalation pressure transducer provides a measurement of the patient's circuit pressure from the inhalation side of the patient's circuit. It is also utilized for detecting of patient circuit occlusions that may occur. While the reference does describe an oxygen source flow sensor within the context of a medical device, the reference does not describe a device or method of using an oxygen source flow sensor to disable potential ignition devices, such as electrocautery.

Another reference, U.S. Pat. No. 8,721,638 [10], describes an electrocautery or laser surgical tool with an integrated gas sensing mechanism which may have the energy output level dictated by the gas sensor. The reference does not describe a device or method of using an oxygen source flow sensor to disable potential ignition devices, such as electrocautery.

Another reference, Barnes et al. (2000) AANA J. 68(2), 153-161 [11], describes an evaluation of oxygen concentration in the microenvironment beneath surgical drapes and evaluating the use of an oxygen scavenging system consisting of a suctioning system beneath the drapes to reduce potential operating room fire risk. Oxygen concentrations were evaluated with a gas analyzer underneath the drapes and at the hypothetical surgical site. The reference does not describe a device or method of using an oxygen source flow sensor to disable potential ignition devices, such as electrocautery.

Another reference, VanCleave, A. M. et al. (2014) Anesth. Prog. 61(4), 155-161 [12], describes the well-known problem of operating room fires. This reference suggests the use of high-volume suction near the potential sources of oxygen as a means to prevent fires upon the contact with an ignition source. The reference does not describe a device or method of using an oxygen source flow sensor to disable potential ignition devices, such as electrocautery.

Another reference, Mehta, S. P. et al. (2013) Anesthesiology 118(5), 1133-1139 [13], describes an analysis of operating room fires. Electrocautery-induced fires during monitored anesthesia care were the most common cause of operating room fires claims. Recognition of the fire triad (oxidizer, fuel, and ignition source), particularly the critical role of supplemental oxygen by an open delivery system during use of the electrocautery, is crucial to prevent operating room fires. The reference recommends continuing education and communication among operating room personnel along with fire prevention protocols in high-fire-risk procedures to potentially reduce the occurrence of operating room fires. The reference does not describe a device or method of using an oxygen source flow sensor to disable potential ignition devices, such as electrocautery.

Another reference, Stewart, et al. (2015) Ophthalmology 122(3), 445-447 [14], describes the recurrence of operating room fires and that it is well known that 3 components are required: fuel (tracheal tubes, drapes, gauze, sponges, solutions containing alcohol or other volatile compounds, patient's hair, gloves, oxygen masks, nasal cannula, dressings, ointments, gowns, gastrointestinal gases, blankets, suction catheters, fiber optic endoscopes, fiber optic cable coverings, and packaging materials); an oxidizing agent (oxygen or nitrous oxide); and an ignition source (electrosurgical or electrocautery devices, lasers, heated probes, drills and burrs, argon beam coagulators, fiber optic light cables, and defibrillator paddles). The reference also describes that different members of the surgical team bear primary responsibility for controlling each: fuel=circulating nurse; oxidizing agent=anesthesiologist; ignition device=surgeon. To avoid the operating room fires, the reference suggests better procedures, appropriate usage of tools and oxygen, and increased communication within the operating room team. The reference does not describe a device or method of using an oxygen source flow sensor to disable potential ignition devices, such as electrocautery.

### Current Invention System

Operating room fires cause significant morbidity to patients and extremely high costs to hospitals. As such, hospitals are directing increasingly more resources toward patient safety and devices in “human factors engineering” to prevent operating room fires from occurring.

Human factors engineering looks at human beings and the systems in which they interact to find ways to improve efficiency, cost, and prevent errors. Operating room fires can occur when the communication to turn off the oxygen prior to electrocautery use, given by the surgeon to the anesthesiologist or staff controlling O₂ flow, is either not given or received; thus, it is an optimal target for human factors engineering. Ever since its release in 1999 by the Institute Of Medicine, there has been a huge push for hospitals to provide resources toward patient safety and to eliminate human factors.

The current invention device may be used in the scenario that is by far the most common scenario for the development of OR fires—for example, patients undergoing head/neck/upper body surgery with electrocautery where oxygen is deployed by nasal cannula, mask, or any other open O2 delivery system. Typically, a surgeon is required to ask the nurse/anesthetist to turn off oxygen before employing cautery. Operating room fires occur when this command is either not given, or not carried out, and electrocautery is deployed while in the presence of oxygen. The present invention contemplates a device that minimizes the threat to the health and safety of patients and medical staff related to these “human factors” by preventing the surgeon from using electrocautery while oxygen is flowing, thus avoiding electrocautery ignition of oxygen in the surgical field.

The device comprises a gas flow sensor connected to a relay switch controlling the electrocautery circuit. Oxygen originating from an anesthesia machine or an O₂ tank runs through the gas flow sensor and continues on to the patient. The flow sensor controls a relay switch state. While O₂ is flowing a relay switch is open and the surgeon is unable to use the electrocautery. After oxygen flow is terminated at the source, there is a short delay in the circuit while flow slows through the meter and O₂ clears from the surgical field. A relay switch is then closed upon reaching a determined threshold flow rate, and the surgeon can safely use the electrocautery. This invention may be easily integrated into pre-existing surgical flow and requires no change in surgeon routine.

The current invention minimizes the impact of human factors on the health and safety of surgical patients and medical staff. An O₂ flow sensor coupled to a relay switch that in turn controls the power to the electrocautery. The flow sensor is between the O₂ tank and patient's nasal cannula and controls the relay switch. The relay switch controls the on/off of the cautery circuit. When O₂ is flowing, the cautery will not turn on. When O₂ flow stops, the relay switch closes allowing use of cautery (after a brief delay to allow O₂ to clear around surgical site).

Electrocautery forceps or surgical tips may plug into the device, and device plugs into cautery unit and receives O₂ from anesthesia machine or patient O₂ tank. Tubing leading distal to the patient's nasal cannula, mask, endotracheal tube, laryngeal mask airway, or other O₂ delivery device is plugged into device O₂ output. This provides a fail-safe for human error and integrates easily into pre-existing surgical flow, without necessitating change in surgeon routine. As it is currently known, no current patents describe a gas flow sensor with a coupled relay switch integrated into the cautery circuit.

In one embodiment, the invention relates to an oxygen sensor system for minimizing the outbreak of an operating room fire comprising: a) an electrocautery device having a proximal end and a distal end for electrically cauterizing tissue; b) an oxygen source comprising an in-line oxygen flow sensor; and c) a relay switch in electrical communication with said oxygen flow sensor and said electrocautery device. In one embodiment, said relay switch is in electrical communication with a control unit. In one embodiment, the invention relates to a method of using the system of described above.

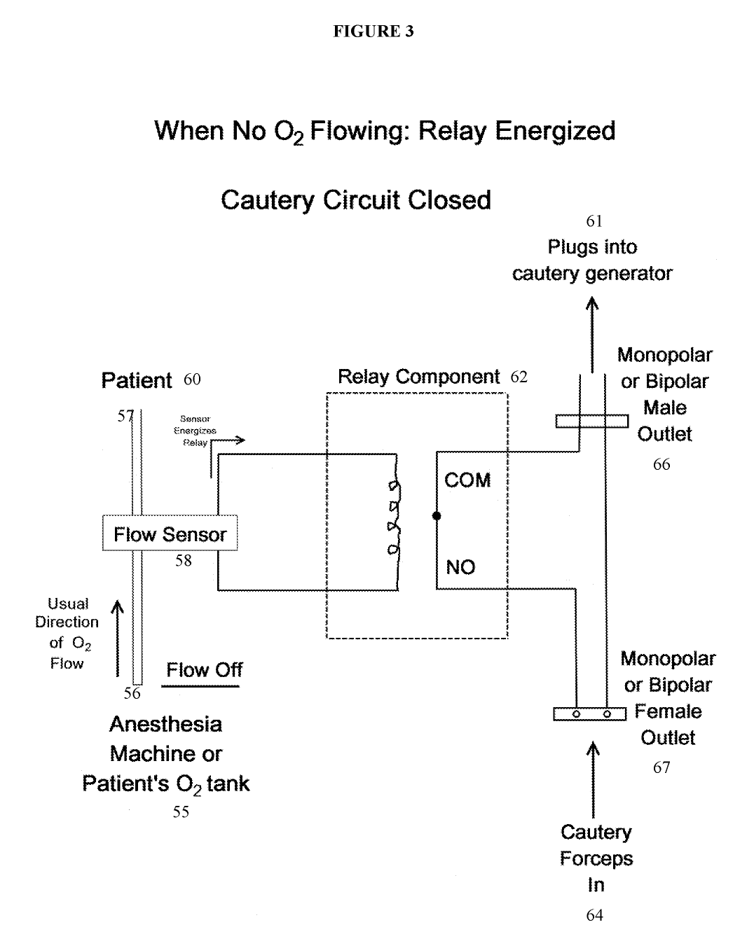

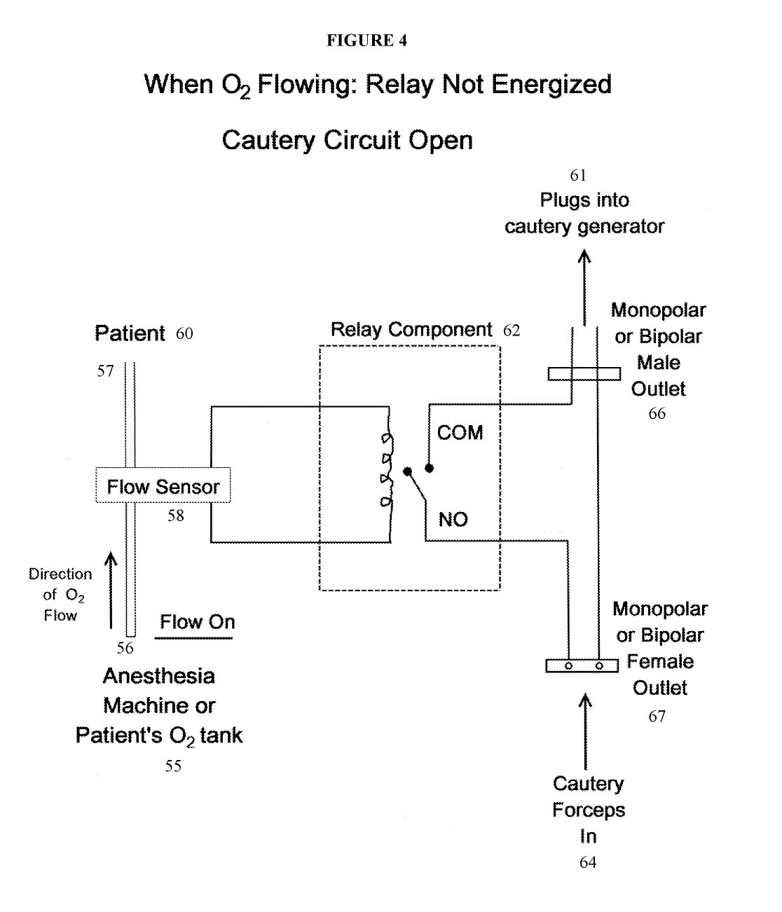

In one embodiment, the invention may be used for monopolar cautery, including monopolar outlets/plugs and circuitry (See FIG. 3 & FIG. 4). The invention may encompass a separate device or a single device with circuitry for both bipolar and monopolar cautery. In one embodiment, the invention further comprises a single device with circuitry for both bipolar and monopolar cautery employs a switch to toggle between said circuitry.

In one embodiment, the invention may further include an indicator on the device signaling a closed cautery circuit, indicating to the surgeon electrocautery is safe to use. In one embodiment, said indicator is a visual indicator. In one embodiment, said visual indicator is an LED. In one embodiment, said indicator is a sonic indicator. In one embodiment, the indicator is a vibration indicator.

In one embodiment, the invention may further include an integrated time delay in the circuit to respond once the oxygen flow reaches a minimum threshold to activate the relay. In one embodiment, the time delay may allow time for oxygen in the surgical field to dissipate.

In one embodiment, the invention may further include additional oxygen concentration sensors integrated into the relay circuitry. In one embodiment, the sensors are clipped under or over surgical drapes monitoring buildup of oxygen concentration. In one embodiment, when the sensor detects concentration above a minimum threshold of oxygen, the sensor deactivates the relay, thus opening the cautery circuit not allowing use of the cautery devices.

Electrocautery Safety Unit Service and Operation

### Overview

The Electrocautery Safety Unit is a medical device that reduces the risk of operating room fires by preventing electrosurgery when elevated oxygen levels are present at the surgery site. This is primarily a concern for facial procedures where oxygen is free to escape into the environment, and the oxygen source and ignition source are in close proximity.

In one embodiment, said system is configured to interface with the internal components of a commercially available device. In one embodiment, said commercially available device comprises a electrocautery generator or electrosurgical generator. In one embodiment, said electrocautery device interfaces with an electrocautery generator and is separately powered through an outlet. In one embodiment, said electrocautery device may interface with an electrocautery generator through an auxiliary port. In one embodiment, said electrocautery device may interface with an electrocautery generator through an expansion port. In one embodiment, said electrocautery device may interface with an electrocautery generator through an existing port. In one embodiment, said electrocautery device utilizes an auxiliary port on an existing electrocautery generator that both powers the device, and receives a signal from said flow sensor or control unit to switch an internal relay or control unit and shut off the ability to cauterize circuits, including both monoploar and bipolar circuits. In one embodiment, said electrocautery device is integrated completely into an electrocautery generator. In one embodiment, the invention may further include an indicator on the device signaling a closed cautery circuit, indicating to the surgeon it is safe to use. In one embodiment, the invention further comprises an indicator that cautery is disabled and one cannot use cautery. In one embodiment, the invention further comprises an indicator that shows the delay time. In one embodiment, said delay time is a number indicator.

### Operation

Although not limiting the current invention to any particular electrocautery generator, one embodiment of the current invention is designed to be used exclusively with a Covidien Force FX electrosurgical generator. In one embodiment, the system consists of a small enclosure housing a flow sensor, a display panel, and control electronics. In one embodiment, the flow sensor is connected in-line with the oxygen supplied from the anesthesia machine to the patient. In one embodiment, a cable on the system controller is connected to an expansion port on the back of the electrosurgical generator via a connector, such as a DE-15 serial connector. In one embodiment, the electrosurgical generator provides power to the system controller, and the system controller sends a disable signal to the electrosurgical generator when it determines that it is unsafe to perform electrosurgery.

In one embodiment, the display panel includes three indicator lights and a numerical display to communicate the device status to operating room personnel. The system operates in the following modes, depending on the measured flow rate, estimated oxygen concentration, and time since the flow rate threshold was last exceeded:

| | Indicator | Numerical | | |
|---|---|---|---|---|
| Mode | Light | Display | Electrocautery | Condition |
| Disabled | Red | Flow Rate | Disabled | Measured flow rate above threshold |
| Ready | Green | Flow Rate | Enabled | Measured flow rate below threshold, Est. |
| | | | | O₂ concentration below safe limit AND |
| | | | | minimum delay time has elapsed* |
| Delay | Yellow | Delay | Disabled | Measured flow rate below threshold, Est. |
| | | Time | | O₂ concentration below safe limit OR |
| | | | | minimum delay time has elapsed* |

*Delay time is measured from last time flow rate was greater than threshold.

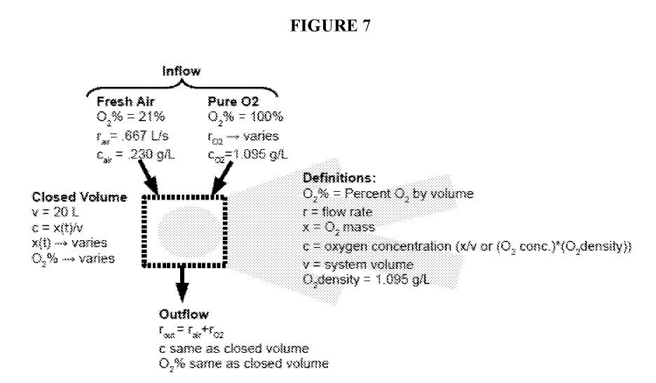

The O₂ concentration is approximated by assuming that the surgery site is a closed volume with a constant inflow of fresh air and a variable inflow of pure O₂. The closed volume is always perfectly mixed, and the outflow is equal to the total inflow. The parameters of the system could be adjusted based on test results to give conservative results for a majority of cases. For the initial prototype a volume of 20 L and a fresh air flow rate of 40 L/min (0.667 L/s) were assumed. The max safe O₂ concentration was assumed to be 24%. See FIG. 7 for a description of the variables considered in the applications of the current invention.

The mass of O₂ in the closed volume, x, is used to find the O₂ concentration by volume. The rate of change of x with respect to time, dx/dt, is equal to the difference in the mass of O₂ entering the system and the mass of O₂ leaving the system, and it can be either positive or negative.

dx/dt=rₐᵢᵣcₐᵢᵣ+r_{O2}c_{O2}−rₒᵤₜcₒᵤₜ (Equation 1)

Since cₒᵤₜ=x/v and rₒᵤₜ=rₐᵢᵣ+r_{O2}, dx/dt can be rewritten as follows. (Because rair, cair, and cO2 are constants, the only variables necessary to calculate dx/dt are r_{O2} and x.)

dx/dt=rₐᵢᵣcₐᵢᵣ+r_{O2}c_{O2}−rₒᵤₜcₒᵤₜ−(r_{O2}+rₐᵢᵣ)*x/v (Equation 2)

The system controller estimates the O₂ concentration by numerically solving this differential equation for x. When the device is first powered on, the initial O₂ concentration is assumed to be equal to normal air, and the mass of O₂ is calculated accordingly. Every 0.25 seconds the O₂ flow rate is measured, and dx/dt is calculated. The change in O₂ mass over this time interval, (dx/dt)*(0.25 s), is added to the previous x value. This new x value is then used to find the O₂ concentration, which the system controller compares to the safe limit.

Because this is a linear first order differential equation, it is also possible to solve for x as a function of time analytically. The solution is:

x=e^{-r}^{ₒᵤₜ}^{t/v}[x₀+(rₐᵢᵣcₐᵢᵣ+r_{O2}c_{O2})/(rₒᵤₜ/v)(e^{r}^{ₒᵤₜ}^{t/v}−1)] (Equation 3)

This can be rearranged to solve for t as a function of x. This solution is what the system controller uses to estimate the delay time remaining for the O₂ concentration to fall below the safe limit.

t=[ln((x−b/a)/(x₀−b/a))/−a] where a=rₒᵤₜ/v and b=rₐᵢᵣcₐᵢᵣ+r_{O}_{₂}c_{O}_{₂} (Equation 4)

### Error Codes

The following error codes are displayed when the flow sensor reading is outside of the expected range:

E-1: Sensor value too high

E-2: Sensor value too low

Once an error code has been displayed, electrosurgery is disabled until the error condition is eliminated and power to the unit is reset.

Error code E-2 is often the result of a backward flow connection.

Hardware Description

Part list

| Item # | Name | Notes |
|---|---|---|
| 1 | Top Cover | Material: 5052 Al, 0.080″ thick. Powder Coat: |
| | | Cardinal T09-BL05 |
| 2 | Front Panel Assembly | |
| 3 | Flow Fitting | M/F Sustarin C M6 green medical grade acetal rod, |
| | | 1.0″ dia |
| 4 | Torx screw | Stainless Stl 6-32x3/8, P/N 92703A253 (McMaster |
| | | Carr) |
| 5 | Back Cover | Material: 6061 Al, 0.5″ thick. Powder Coat: Cardinal |
| | | T09-BL05 |
| 6 | Grommet | |
| 7 | Foot | P/N 9540K664 (McMaster Carr) |
| 8 | Cat 5 Cable | P/N CAB008-MP-L5-BK (IEC, 303-288-5000) |
| 9 | #10-32 socket cap screw | |
| 10 | Bottom plate | Material: 6061 Al, 0.125″ thick. Powder Coat: |
| | | Cardinal T09-BL05 |
| 11 | P4 DB15 HD Male | P/N DH15MS (IEC, 303-288-5000) |
| | Connector | |
| 12 | D-sub hood | P/N DB09HFL (IEC, 303-288-5000) |
| 13 | U3 Flow Sensor | P/N D6F-20A5-000 (Omron) |
| 14 | Flow Sensor Cable | P/N D6F-CABLE1 (Omron) |
| 15 | Connector, 3 pin | P/N 50-37-5033 (Molex) |
| 16 | Connector crimp terminal | P/N 08-70-1040 (Molex) |
| 17 | Connector, 5 pin | P/N 50-37-5053 (Molex) |
| 18 | Strain relief fitting | From 3/8″ cable grip electrical fitting (Home Depot) |
| 19 | 6-32 Socket Cap Screw | |
| 20 | #6 Flat washer | |
| 21 | Nut Plate | P/N 98001A110 (McMaster Carr) |
| 22 | M3 screw | Max torque .59 N * m per sensor data sheet |
| 23 | M3 nut | |
| 24 | M3 flat washer | |
| 25 | M3 lock washer | |
| 26 | O-ring | #10, Viton, P/N 2-010-FKM-75 (Rocket Seals Corp., |
| | | 303-777-7024) |
| 27 | Front panel graphic | |
| | overlay | |
| 28 | Front panel aluminum | Material: 6061 Al, 0.5″ thick |
| | plate | |
| 29 | Indicator light tinted outer | |
| | lens | |
| 30 | Indicator light diffuser lens | |
| 31 | Display tinted outer lens | |
| 32 | Display text mask | Digital printed clear adhesive vinyl, graphics file |
| | | SB07B |
| 33 | Text diffuser lens | |
| 34 | Text gray lens | Used to adjust brightness of text |
| 35 | Circuit Board Assembly | |
| 36 | #4-40 Screw | |
| 37 | Plastic flat washer | .115″ ID, .250″ OD, .063″ thick |
| 38 | Printed Circuit Board | P/N SB04B-1 |
| 39 | U2 4-digit display board | P/N COM-11629 (Sparkfun Electronics) |
| 40 | Breakaway male header | |
| 41 | D4-D15 White LED, 3 mm | P/N K/LED30W (Velleman) |
| 42 | LED spacer | P/N SB08B 3d printed part |
| 43 | D1 Red LED, 5 mm | P/N 276-0309 (Radio Shack) |
| 44 | D2 Yellow LED, 5 mm | P/N 276-0351 (Radio Shack) |
| 45 | D3 Green LED, 5 mm | P/N 276-0304 (Radio Shack) |
| 46 | U1 Microcontroller board | Arduino Nano 3.0 or equivalent |
| 47 | Female header connector | P/N PRT-00115 (Sparkfun Electronnics) |
| 48 | J1, J2 5 pin header | P/N 22-03-5055 (Molex) |
| | connector | |
| 49 | J3 3 pin header connector | P/N 22-03-5035 (Molex) |
| 50 | RN1 Resistor network | P/N 4114R-2-152LF (Bourns), 13 × 1.5 ohm |
| 51 | R2-R4 resistor | 330 ohm, 1/4 W |
| 52 | IC1 Voltage reference, | P/N LM4040AIZ-5.0/NOPB (Texas Instruments) |
| | 5.0 v | |
| 53 | R1 Resistor | 10k ohm, . W |

### LIST OF ADDITIONAL REFERENCE NUMERALS

54 device
55 oxygen source
56 oxygen flow in
57 oxygen flow out
58 oxygen flow sensor
59 nasal cannula, mask, endotracheal tube, laryngeal mask airway, or other O₂ delivery device
60 patient
61 electrocautery generator, power source/cautery unit
62 relay switch
63 electrocautery devices, forceps, surgical tip
64 electrocautery device connection into the relay switch device
65 cautery circuit
66 relay switch device monopolar/bipolar male outlet
67 relay switch device monopolar/bipolar female outlet

### Example of Microcontroller Code for One Embodiment:

Thus, specific systems, devices and methods of an operating room fire prevention and electrocautery safety device have been disclosed. It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### REFERENCES

1. Committee, A. T. et al. (2013) “Electrosurgical Generators,” Gastrointest. Endosc. 78(2), 197-208.
2. Ogg, M. and King, C. A. (2009) “Recommended Practices for Electrosurgery,” 2012 Perioperative Standards and Recommended Practices, 99-118.
3. Cohen, J. (2014) “Argon Plasma in the Management of Gastrointestinal Hemorrhage,” UpToDate.
4. Gedebou, T. “Oxygen Sensory System That Minimizes Outbreak of Operating Room Fires,” United States Patent Application Publication Number US 2006-0058784 A1, application Ser. No. 10/940,330, filed Sep. 14, 2004. (published Mar. 16, 2006).
5. Foltz, J. W. and Lubin, M. E. “Electrical Cautery-Oxygen Safety Device,” U.S. Pat. No. 7,296,571, application Ser. No. 10/856,205, filed May 27, 2004. (issued Nov. 20, 2007).
6. Gedebou, T. “Comprehensive System That Minimizes Outbreak of Operating Room Fires,” United States Patent Application Publication Number US 2006-0069387 A1, application Ser. No. 10/949,856, filed Sep. 24, 2004. (published Mar. 30, 2006).
7. Oberle, M. D. and Probert, J. C. “Pulse Width Modulated Medical Gas Concentration Control,” U.S. Pat. No. 8,789,524, application Ser. No. 12/017,815, filed Jan. 22, 2008. (issued Jul. 29, 2014).
8. Wenrich, M. S. “Organ Preservation Apparatus and Methods,” United States Patent Application Publication Number US 2009-0291486 A1, application Ser. No. 12/535,103, filed Aug. 4, 2009. (published Nov. 26, 2009).
9. Woodring, P. L. et al. “Medical Ventilator,” United States Patent Application Publication Number US 2003-0062045 A1, application Ser. No. 10/295,141, filed Nov. 15, 2002. (published Apr. 3, 2003).
10. Deutscher, E. M. and Vassallo, F. A. “Gas Sensing Surgical Device and Method of Use,” U.S. Pat. No. 8,721,638, application Ser. No. 13/644,202, filed Oct. 3, 2012. (issued May 13, 2014).
11. Barnes, A. M. and Frantz, R. A. (2000) “Do Oxygen-Enriched Atmospheres Exist beneath Surgical Drapes and Contribute to Fire Hazard Potential in the Operating Room?,” AANA J. 68(2), 153-161.
12. VanCleave, A. M. et al. (2014) “The Effect of Intraoral Suction on Oxygen-Enriched Surgical Environments: A Mechanism for Reducing the Risk of Surgical Fires,” Anesth. Prog. 61(4), 155-161.
13. Mehta, S. P. et al. (2013) “Operating Room Fires: A Closed Claims Analysis,” Anesthesiology 118(5), 1133-1139.
14. Stewart, M. W. and Bartley, G. B. (2015) “Fires in the Operating Room: Prepare and Prevent,” Ophthalmology 122(3), 445-447.

We claim:

1. An oxygen sensor system for minimizing the outbreak of an operating room fire comprising:
a) an electrocautery device having a proximal end and a distal end for electrically cauterizing tissue;
b) an oxygen source comprising an in-line oxygen flow sensor; and
c) a relay switch in electrical communication with said oxygen flow sensor and said electrocautery device.

2. The system of claim 1, wherein said relay switch is in electrical communication with a control unit.

3. A method of using the system of claim 1.

4. The system of claim 1, wherein said electrocautery device comprises monopolar cautery.

5. The system of claim 1, wherein said electrocautery device comprises bipolar cautery.

6. The system of claim 1, wherein said sensor system with said relay switch comprises a single device with circuitry for both bipolar and monopolar cautery.

7. The system of claim 6, wherein said relay switch further comprises a switch to toggle between said bipolar and monopolar cautery circuitry.

8. The system of claim 1, wherein said system further comprises an indicator on the device signaling a closed cautery circuit.

9. The system of claim 8, wherein said indicator is a visual indicator.

10. The system of claim 9, wherein said visual indicator is an LED.

11. The system of claim 8, wherein said indicator is a sonic indicator.

12. The system of claim 8, wherein said indicator is a vibration indicator.

13. The system of claim 1, wherein said system further comprises an integrated activation time delay in the relay switch after sensing a minimum threshold of oxygen to activate the relay.

14. The system of claim 1, wherein said system further comprises additional oxygen concentration sensors integrated into the relay circuitry.

15. The system of claim 14, wherein said sensors are clipped under or over surgical drapes monitoring buildup of oxygen concentration.

16. The system of claim 14, wherein said at least one sensor detects concentration above a minimum threshold of oxygen, the sensor deactivates the relay, thus opening the cautery circuit not allowing use of the cautery devices.

17. The system of claim 16, wherein said minimum threshold of oxygen comprises 25%.

18. The system of claim 1, wherein said electrocautery device interfaces with an electrocautery generator and is separately powered through an outlet.

19. The system of claim 1, wherein said electrocautery device interfaces with an electrocautery generator through an auxiliary port.

20. The system of claim 1, wherein said electrocautery device utilizes an auxiliary port on an existing electrocautery generator that both powers the device, and receives a signal from said flow sensor to switch an internal relay and shut off the ability to cauterize both monoploar and bipolar circuits.

21. The system of claim 1, wherein said electrocautery device is integrated completely into an electrocautery generator.
